(19) United States
(12) Patent Application Publication
(10) Pub. No.: US 2011/0229530 A1
(43) Pub. Date: Sep. 22, 2011

(54) PHARMACEUTICAL DOSAGE FORMS FOR TIME-SPECIFIC DRUG DELIVERY

(76) Inventors: Andrea Gazzaniga, Santa Giulietta (IT); Matteo Cerea, Grumello Del Monte (IT); Alberto Cozzi, Busto Arsizio (IT); Anastasia Anna Foppoli, Milano (IT); Maria Edvige Sangalli, Pavia (IT); Gianaldo Tavella, Pavia (IT); Lucia Zema, Como (IT)

(21) Appl. No.: 13/055,191

(22) PCT Filed: Jul. 23, 2009
(86) PCT No.: PCT/EP2009/005370

§ 371 Date: Mar. 25, 2011

(30) Foreign Application Priority Data
Jul. 24, 2008 (IT) MI2008A001351

(51) Int. Cl. A61K 9/00; A61K 31/167; A61P 29/00; B29C 47/20

(52) U.S. Cl. 424/400; 514/630; 264/541

(57) ABSTRACT

The present invention refers to an oral dosage form intended for the time-controlled release of drugs comprising a container for one or more active ingredients optionally in admixture with pharmaceutically acceptable excipients, said container consisting of at least two parts or elements that can be joined together so as to seal the contents, at least one of said elements being composed of one or more hydrophilic polymers that undergo a decrease in the glassy-rubbery transition temperature when in contact with aqueous fluids, except for polymers with pH dependent solubility soluble only at pH values above 5, optionally in admixture with pharmaceutically acceptable excipients, the wall of the elements of the container being of such thickness as to delay the release of the contained drug with respect to the time of administration.

The present invention concerns pulsatile release pharmaceutical dosage forms meant for the therapy of pathologies with symptoms recurring at night or in the early morning hours.

### STATE OF THE ART

Pathologies for which the treatment with pulsatile release systems may be significantly important (angina, hypertension, rheumatoid arthritis, early morning insomnia and other) are among the most common (in the world there are 972 million people suffering from hypertension, Hajjar I. et al., Annu. Rev. Public Health 27, 465-90 (2006); only in the North America 20% of the population suffer from sleep disorders, Young T. et al., Am. J. Respir. Crit. Care Med. 165, 1217-1239 (2002)).

When the symptoms recur during the night or early morning hours, the treatment of the previously mentioned pathologies is performed by administering prolonged release systems with constant drug liberation that generally release the active ingredient during the entire period of sleep. Alternatively, conventional dosage forms are administered during the night hours therefore requiring the interruption of normal sleeping patterns.

In the literature numerous examples of systems for the treatment of the above described pathologies may be found. These systems have never been used in therapy and consist of drug-containing solid cores such as tablets, hard capsules, softgels and pellets, mainly with immediate release behavior, provided with coatings able to delay the penetration of biological fluids and therefore controlling the onset of release and eventually drug absorption.

Examples of systems with a pulsatile release of the active ingredient are those in which the coating layer consists of a pH-dependent polymeric layer soluble at pH values above 5, also named delayed release systems. These systems, administered orally, remain intact during the time of residence in the stomach, where the pH is around 1-3. After passing the pyloric valve, the system finds a physiological environment with a higher pH (Evans D. F. et al., Gut 29, 1035-1041 (1988)), allowing the protective layer to dissolve and the drug to be released.

In this type of approach, the drug release latency is determined by the site where the dosage form is located. For this reason, the release performances of these systems are subjected to the variability of gastric emptying time, which in turn is dependent on food intake conditions.

Other examples of pulsatile release dosage forms proposed in the literature are those coated with hydroxypropyl methylcellulose (HPMC), hydroxypropyl cellulose (HPC), hydroxypropyl ethylcellulose (HEC), or a mixture of waxes and surfactants, which are applied to the dosage form by double compression, film coating or powder layering (Gazzaniga A. et al., Eur. J. Pharm. Biopharm. 40, 246-250 (1994); Gazzaniga A. et al., S.T.P. Pharma Sci. 5, 83-88 (1995); WO9832425 (1998); ES2232886T (2005); Matsuo M. et al., Chem. Pharm. Bull. 43, 311-314 (1995); Fukui E. et al., J. Control. Release 68, 215-223 (2000); Bachwal A. R., U.S. Pat. No. 0,118,267 (2005); DE 4122039 (1992)), and finally osmotic pumps.

A complete review on the time-dependent pulsatile release dosage forms published so far in the scientific literature is described by Maroni A. et al., Expert Opin. Drug Deliv. 2, 855-871(2005), whereas a review on the chronopathologies is reported in Smolensky M. H., Peppas N. A., Adv. Drug Deliv. Rev. 59, 823-824 (2007).

Several patents and patent applications concern the preparation and the use of capsules as pharmaceutical dosage forms, for both immediate and controlled release.

For example, EP 1258242 describes a process aimed at obtaining capsules without describing the relevant release properties, whereas U.S. Pat. No. 5,674,530 describes the preparation of capsules for drug release without any reference to a time-dependent delayed release.

The use of starch capsules for drug release is described by Vilivalam V. D. et al., Pharm. Sci. Tech. Today 3, 64-69 (2000).

US 2001/036473 and WO 00/18377 disclose the preparation of a system for the delivery of drugs either to the small intestine or the colon comprising an immediate release capsule composed of materials different from gelatine, such as HPMC. Said capsule is provided with a coating for the attainment of enteric and colonic release of drugs. The coating is based on a typical gastric resistant polymer (such as Eudragit L30D-55, polyvinylacetate phthalate (PVAP), cellulose acetate phthalate (CAP)) with pH dependent solubility, soluble at pH values above 5.5, applied in different amounts ranging from 5 mg/cm2 to 10 mg/cm2.

Ogura T. et al., Pharm Tech. Europe 11, 32-42 (1998) demonstrates the possibility of substituting gelatine with non-animal derivatives such as HPMC and small amounts of carrageenan as preparation materials for conventional hard capsules. As a matter of fact, capsule shells made from gelatine contain 13-15% water and therefore are not suitable for hydrolysable drugs, as some may react with the amino groups of gelatine causing discoloration or cross-linking and undesired delay in shell disintegration time.

U.S. Pat. No. 4,917,885 describes hard capsules prepared with water soluble cellulose ethers, such as alkyl cellulose, hydroxyalkyl cellulose, alkyl hydroxyalkyl cellulose and polyvynil alcohol (PVA) in weight ratio of 70:30 to 98:2. The described capsules are claimed to have a remarkably low permeability to oxygen and moisture thus protecting the contained drug substance from their influence. The capsules in U.S. Pat. No. 4,917,885 are prepared by dipping a preheated mandrel pin into an aqueous polymeric solution and subsequently drying the solution on the surface of the pin which forms a crust of the polymeric composition. The crust is then removed from the pin to form the capsule shell. Preliminary tests were performed in order to evaluate the polymeric blends by studying isolated films 100 microns thick. Capsules prepared with different polymeric blends demonstrated to have disintegration times ranging from 6.5 to 7.0 minutes.

U.S. Pat. No. 6,228,396 refers to a pharmaceutical composition in capsular form for specific colonic release. U.S. Pat. No. 5,788,987 describes a method for the preparation of a system intended for the treatment of chronopathologies.

US 2005/249807 provides pharmaceutical compositions for preparing, by an injection molding technique, capsule shells, linkers, spacers, multicomponent capsules and pharmaceutical dosage forms designed for the sustained, delayed or pulsed release of drugs. The invention describes a composition for the preparation of the capsules shell and linkers preferably including percentages of 10-80% methacrylic acid copolymers, poly (meth)acrylate copolymer and ammonium methacrylate copolymers such as Eudragit RL 100 or RS 100 and percentages from about 30 to about 70% w/w of thermoplastic materials such as HPC, as well as the addition of a lipophilic lubricant such as stearyl alcohol, a surfactant such as Tween 80, plasticizers such as TEC, ATEC, DBS or PEG, dissolution modifying agents such as mannitol, lactose, HPC, HPMC, sugars, citric acid and disintegrants.

WO 2004/010978 describes the preparation of capsules by injection molding for pulsatile drug release using gastric resistant polymers.

The solutions proposed so far for the therapy of chronopathologies are based on drug containing cores (tablets, capsules, pellets) coated with materials able to slow down the penetration of aqueous fluids and therefore delay the interaction of these with the cores. For the majority of the presented systems, a coating process is required and the times involved could vary based on the chosen technology.

In the described systems which involve pH-dependent polymers, as previously discussed, there is no time-control of the drug release but more a site-control, with poor intra- and inter-subject reproducibility of drug release due to the variability of gastric emptying time.

In the examples in which the use of insoluble polymers are described, the lag phase involves an important diffusion phase, and the drug is subsequently delivered with prolonged release kinetics. The presence of insoluble materials in the coating or in the capsule shell is often claimed to confer rigidity and mechanical strength to the structure, but it also involves a control of the release rate which is not observed when only soluble materials are employed. When insoluble polymers such as poly (meth)acrylate copolymer and ammonium methacrylate copolymers (Eudragit RL 100 or RS 100), ethyl cellulose or cellulose acetate phthalate are included in the coating or in the capsule shell, the process of dissolution and mechanical erosion of the swollen material are hindered or impaired and permeation as well as diffusion phenomena of the drug through the film or shell are more significant. In addition, the use of insoluble materials such as Eudragit RL or RS in the composition of the coating or the capsule shells requires the elimination of insoluble residues with the faeces thus raising patient acceptability issues and eventually a poor compliance with the therapy. These problems can further be complicated by the use of lipophilic materials (e.g. stearyl acid), as lubricants in pharmaceutical processes. Finally, the use of insoluble materials such as Eudragit RL and RS could impart to the release performances of coated systems or capsule shells a high variability depending on the hydrodynamic conditions.

In addition, some of the technologies proposed require complex and expensive preparation processes which can hardly be scaled-up for industrial production.

### DESCRIPTION OF THE INVENTION

The present invention provides an oral pharmaceutical dosage form for the pulsatile release of drugs consisting of containers including at least two parts prepared with materials able to delay the release of the contents for a programmable period of time.

The container can deliver the drug in solid, liquid, or semi-solid form, optionally in admixture with other pharmaceutical active ingredients and/or suitable excipients, or previously formulated as powders, capsules, tablets, granulate, pellets, nano- or micro-particles.

The two parts of said container are modeled so that two units are formed and assembled in order to contain a pharmaceutical formulation. The employed materials are able to delay the contact of the biological fluids with the contents for a time period that can be modulated as a function of the thickness of the container shell.

At least one of the assemblable parts or elements of the container, which fit together tightly, is prepared with one or more hydrophilic polymers which undergo a decrease in the glassy-rubbery transition temperature when in contact with aqueous fluids, with the exception of polymers with pH-dependent solubility and soluble at pH values above 5, optionally in admixture with pharmaceutically acceptable excipients. Polymers with pH-dependent solubility and soluble at pH values above 5 are those typically used for preparation of enteric film coatings, also known as gastro-resistant, such as methacrylic acid copolymers (Eudragit® L 100, Eudragit® L 12.5, Eudragit®L 12.5 P, Eudragit® L 100-55, Eudragit® L 30 D-55, Eudragit® S 100, Eudragit® S 12.5, Eudragit® S 12.5 P, Eudragit® FS 30 D), hydroxypropyl methylcellulose phthalate (HPMCP), hydroxypropyl methylcellulose acetate succinate (HPMCAS), polyvinylacetate phthalate (PVAP), cellulose acetate phthalate (CAP).

In the present invention, the containers are capable of delaying drug release regardless of the presence or absence of an outer gastric resistant film.

In a preferred configuration of the invention, both container parts are prepared with the same polymer, being the two parts assembled as a capsule with substantially rigid shells, with thickness greater than 350 microns, preferably between 500 and 1500 microns.

The polymer used for the preparation of the container is chosen among HPMC, HPC, HEC, vinyl copolymers such as polyvinyl pyrrolidone (PVP) and polyvinylalcohol (PVA), polyethylenglycols (PEG), polyethylene oxides (poloxamers, PEO), alginic acid derivates, and mixtures thereof.

Cellulose ethers such as HPMC and HPC are particularly preferred.

The present dosage forms can be prepared by conventional techniques such as, for example, depending on the types of material, extrusion, in such a way as to form the elements which are joined together by snapping or welding so as to seal the contents.

Said welding can be accomplished by applying a polymeric solution in suitable solvents or by fusion of the contact surface induced by ultrasounds or heat transmitted by contact or by irradiation.

Alternatively, the two parts of the container can be prepared from films molded to obtain a cavity in which one or more optionally formulated active ingredients are loaded, of which a delayed release is desired. Said films are obtained by material layering from solutions or dispersions onto inert substrates and subsequent solvent removal (casting), or by forcing molten and/or softened masses and/or kneaded products prepared with appropriate binding agents through a calender.

The container can also be obtained by forcing molten and/or softened masses and/or kneaded products obtained with appropriate binding agents into shaped dies (injection molding).

Finally, the container can be prepared by modeling molten and/or softened masses and/or kneaded products prepared with appropriate binding agents as a result of the pressure exerted by two optionally heated dies.

Filling of the container can be performed by conventional equipment used for hard gelatine capsules. If desired, part of the drug or a drug different from the one inserted in the container can be included in the composition of the shell.

The dosage forms described in the present invention allow the drug to be released after a programmable time period following oral administration.

Therefore, said dosage forms are particularly indicated for the therapy and/or the prevention of chronopathologies (pathologies with symptoms prevailingly recurring at particular times of the day or of the night).

The invention provides the tools for the delivery of drugs prescribed for therapies in which a lag phase is desired after the administration of the dosage form, followed by a release phase that can be immediate or prolonged depending on the therapeutic needs. In particular, the invention is exploitable for those pathologies in which the symptoms are exhibited predominantly during the night or in the early morning hours, allowing the absorption of the active ingredient and the appearance of the symptoms to be aligned in order to increase the therapeutic efficacy and reduce side effects.

Examples of such pathologies comprise bronchial asthma with prevalent dyspnoic attacks during the night hours, sleep disorders such as early morning insomnia, rheumatoid arthritis and cardiovascular pathologies with prevalent incidence in the early morning hours.

The invention offers the possibility of obtaining delays in drug release regardless of the gastrointestinal transit of the system (pH-independent behavior). The delays can be programmed as a function of the thickness and composition of the container shell. The system is characterized by a great versatility. In addition, being the container developed separately from the formulation to be delivered, a wide array of systems can be available among which the most suitable one may be selected for the therapeutical needs and lag time required. The release of the conveyed drug is delayed for at least one hour in vivo, preferably two.

The dosage form described in the present invention can be coated with various materials and by different techniques. When the coating is prepared with pH-dependent solubility polymers, in particular soluble at pH values above 5, the dosage form can be used for oral colon delivery of drugs based on a time-dependent formulation approach (A. Maroni et al., Expert Opin. Drug Deliv.: 2(5), 855-871 (2005)). Colonic drug delivery is advantageous not only for the therapy and prevention of pathologies affecting the large intestine (ulcerative colitis, Crohn's disease, colorectal adenocarcinoma, microflora alterations), but also for pharmacological treatments that require a systemic absorption of the drug.

The present invention offers the following advantages with respect to the existing technologies:
Versatility in terms of filling with different drugs and/or formulations;
Possibility of obtaining programmable lag phases as required by therapeutic needs;
Possibility of filling using the same equipment as the one used for the preparation of hard gelatine capsules;
Short production time, compatible with industrial scale-up;
Short time required for development of delayed release dosage form (without the need of developing coating technologies with possible compatibility problems);
Use of GRAS (Generally Recognized as Safe) materials.

Furthermore, it is important to underline that the capsules described in EP 1258242, U.S. Pat. No. 5,674,530, Ogura T. et al., Pharm Tech. Europe 11, 32-42 (1998), U.S. Pat. No. 4,917,885 and US 2001/036473, are intended for an immediate drug release. Therefore, delay phases possibly observed in the relevant release profiles due to the time required for hydration of the capsule-forming materials are considered unacceptable as they would impair the intended prompt drug liberation. On the contrary, in the present invention the delay is deliberately pursued through the use of suitable materials and the selection of the appropriate shell thickness in order to obtain modified-release dosage forms.

In addition, as compared with the capsules prepared according to US 2005/249807, the device described in the present invention releases “in vitro” the model drug with significantly shorter diffusion phases. Indeed, a prompt and complete release of the drug is accomplished in about 15-30 minutes. Moreover, in the present invention, the use of insoluble materials is avoided. Completely soluble capsules are thus obtained. This innovation overcomes the drawbacks associated with the presence of insoluble residues in the faeces, which may raise patient compliance issues. The following examples describe the invention more in detail.

### BRIEF DESCRIPTION OF THE FIGURES

FIG. 1: In vitro release profiles of acetaminophen obtained from dosage forms described in Example 1;

FIG. 2: In vitro release profiles of acetaminophen obtained from dosage forms described in Example 2;

FIG. 3: In vitro release profiles of acetaminophen obtained from dosage forms described in Example 3;

FIG. 4: In vitro release profiles of acetaminophen obtained from dosage forms described in the Example 4;

FIG. 5: Schematic diagram of the capsule prototype described in Example 5;

FIG. 6: In vitro release profiles of acetaminophen obtained from dosage forms described in Example 5;

FIG. 7: Salivary concentration profiles of acetaminophen obtained from dosage forms described in Example 6;

FIG. 8: Salivary concentration profiles of acetaminophen obtained from dosage forms described in Example 7.

### EXAMPLE 1

### Composition

| | |
|---|---|
| HPMC K100LV | 21.4% |
| HPC LV | 50.0% |
| PEG 400 | 7.1% |
| H₂O | 21.4% |

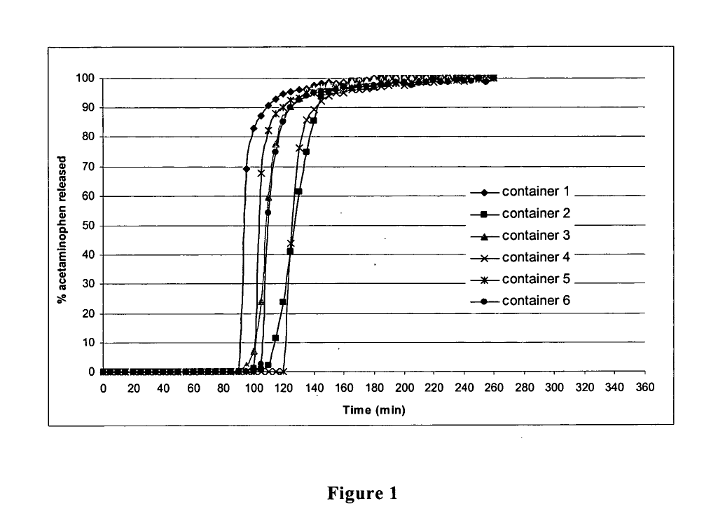

The mixture was kneaded in a high shear mixer. The polymeric dough was stored in a closed container for 24 hours prior to the extrusion process. For the extrusion, a single screw extruder was employed and cylindrical rods (8 mm diameter) were prepared. The rods were forced through a calender in order to prepare a film with thickness between 350 and 1500 microns. After drying, the films were cut with a die and shaped with molds heated at about 130° C. fixed on a manual press to form the elements of the container. Once the two parts were prepared, 200 mg of acetaminophen were filled into the container cavity. The system was closed by applying an HPMC aqueous solution onto the contact surface between the elements. The release profiles obtained from systems prepared by this method are reported in FIG. 1 (disintegration apparatus with sinkers, deionized water, 800 mL, 37° C., n=6).

### EXAMPLE 2

### Composition

| | |
|---|---|
| HPMC K100LV | 28.6% |
| HPC LV | 42.8% |
| PEG 400 | 7.1% |
| H₂O | 21.4% |

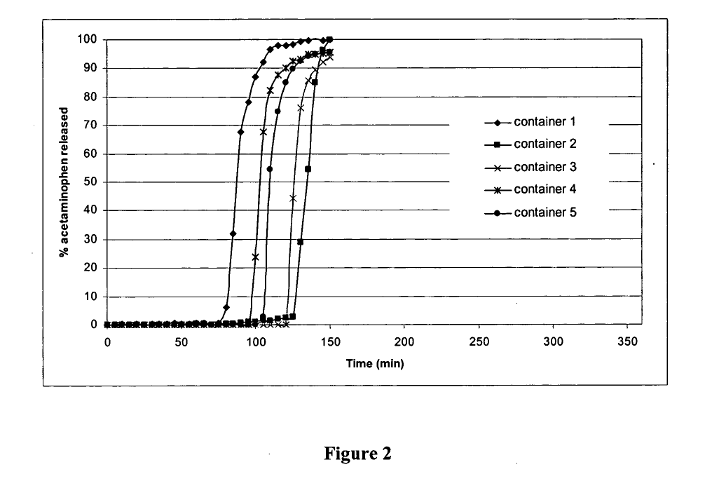

The mixture was kneaded in a high shear mixer. The polymeric dough was stored in a closed container for 24 hours prior to the extrusion process. Extrusion by a single screw extruder was performed and cylindrical rods (8 mm diameter) were prepared. The rods were forced through a calender in order to prepare a film with thickness between 350 and 1500 micron; After drying, the films were heated at about 130° C. with a hot air gun and shaped with molds at room temperature fixed on a manual press to form the elements of the container. Once the two parts were prepared, 200 mg of acetaminophen were filled into the container cavity. The system was closed and cut by applying a tube die heated at 130° C. The release profiles obtained from systems prepared by this method are reported in FIG. 2 (disintegration apparatus with sinkers, deionized water, 800 mL, 37° C., n=5).

### EXAMPLE 3

### Composition

| | |
|---|---|
| HPC LF | 95% |
| PEG 1500 | 5% |

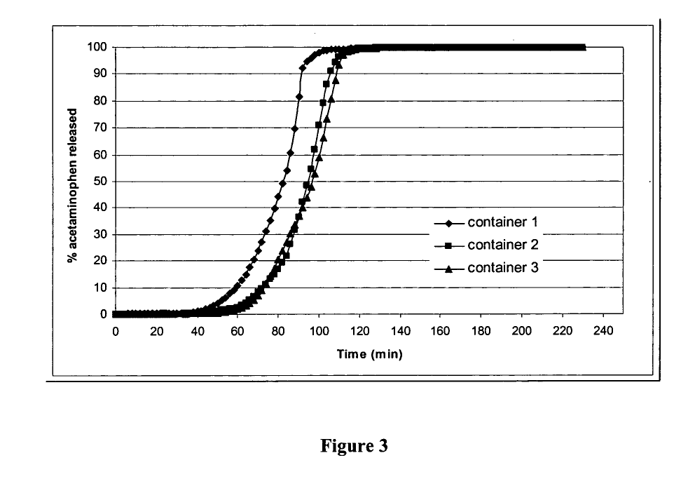

The mixture was prepared in a planetary mixer at a rotating rate of 68 rpm for 10 min and loaded into the hopper of a single screw extruder equipped with a screw with compression ratio of 19:25 D and with a 10 mm diameter die. The temperatures were set at T1:150° C., T2:155° C., T3:160° C. e T4:150° C., rotating speed was 15 rpm. The hot rod at the exit of the extruder was forced through the rolls of a calender with a 1 mm gap. The obtained films were shaped with molds heated at about 130° C. fixed on a manual press to form the elements of the container. Once the two parts were prepared, 200 mg of acetaminophen were filled into the container cavity. The system was closed by applying an HPMC aqueous solution onto the contact surface between the elements. The release profiles obtained from systems prepared by this method are reported in FIG. 3 (disintegration apparatus with sinkers, deionized water, 800 mL, 37° C., n=3).

### EXAMPLE 4

### Composition

| | |
|---|---|
| HPC JF | 95% |
| PEG 1500 | 5% |

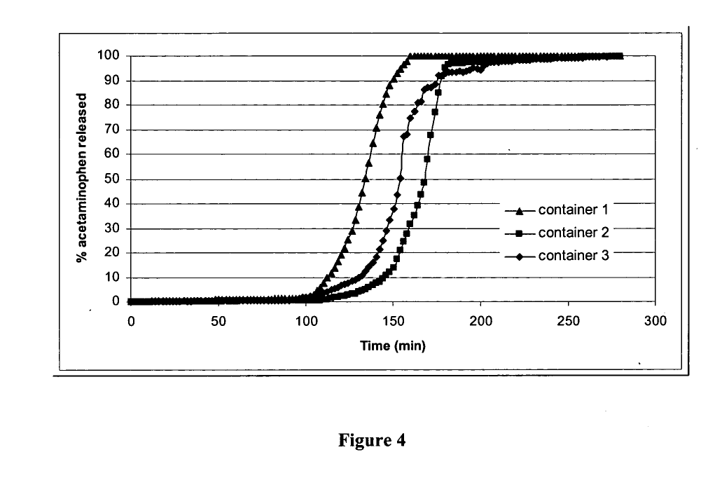

The mixture was prepared in a planetary mixer at a rotating rate of 68 rpm for 10 min and loaded into the hopper of a single screw extruder equipped with a screw with compression ratio of 19:25 D and with a 10 mm diameter die. The temperatures were set at T1:150° C., T2:155° C., T3:160° C. and T4:150° C., rotating speed was 15 rpm. The hot rod at the exit of the extruder was forced through the rolls of a calender with a 1 mm gap. The obtained films were shaped with molds heated at about 130° C. fixed on a manual press to form the elements of the container. Once the two parts were prepared, 200 mg of acetaminophen were filled into the container cavity. The system was closed by applying an HPMC aqueous solution onto the contact surface between the elements. The release profiles obtained from systems prepared by this method are reported in FIG. 4 (disintegration apparatus with sinkers, deionized water, 800 mL, 37° C., n=3).

### EXAMPLE 5

### Composition

| | |
|---|---|
| HPC LF | 90% |
| PEG 1500 | 10% |

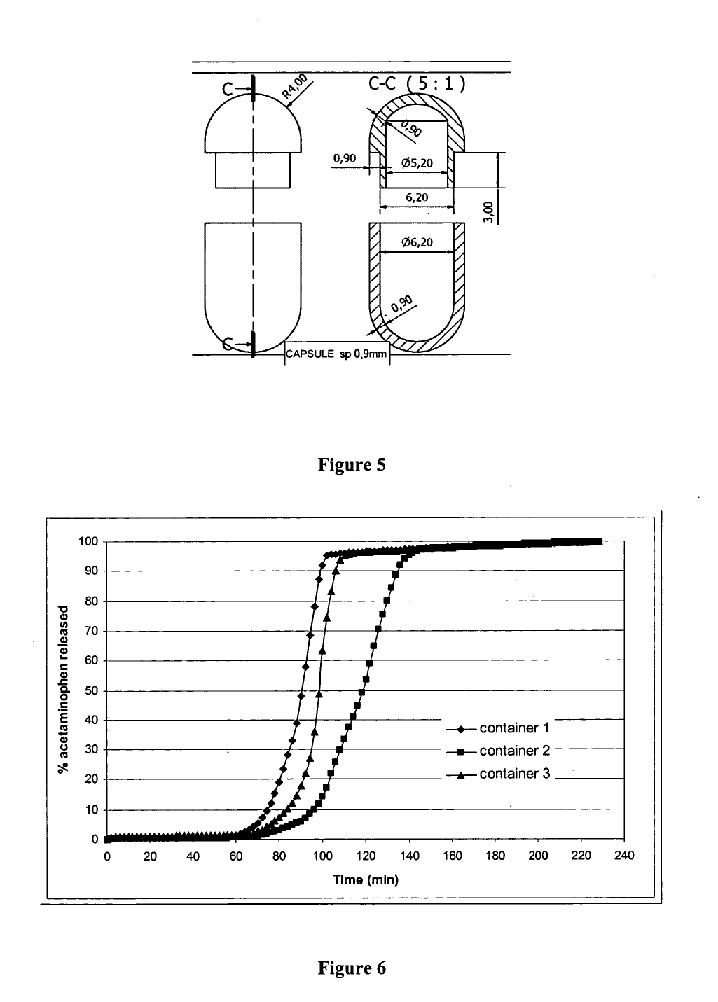

The elements of the container were obtained by loading the powder mixture previously prepared in a planetary mixer into an injection molding press (Baby Plast 6/10P, Cronoplast SI). The powder substrate was fed from the hopper into the plasticization unit (165° C.) and then conveyed to the injection unit (160° C.), where it was press-injected through a 2 mm diameter nozzle (160° C.) into the mold cavity within a defined period of time. Before product ejection, the mold was kept closed in order to allow the processed material to cool down and harden. A schematic diagram of the mold is reported in FIG. 5. Once the two parts were prepared, 200 mg of acetaminophen were filled into the container cavity. The system was assembled and sealed by applying an HPMC aqueous solution onto the contact surface between the elements. The release profiles obtained from systems prepared by this method are reported in FIG. 6 (disintegration apparatus with sinkers, deionized water, 800 mL, 37° C., n=3).

### EXAMPLE 6

### Composition

| | |
|---|---|
| HPC LF | 90% |
| PEG 1500 | 10% |

The elements of the container were obtained by loading the powder mixture previously prepared in a planetary mixer into an injection molding press (Baby Plast 6/10P, Cronoplast SI). The powder substrate was fed from the hopper into the plasticization unit (165° C.) and then conveyed to the injection unit (160° C.), where it was press-injected through a 2 mm diameter nozzle (160° C.) into the mold cavity within a defined period of time. Before product ejection, the mold was kept closed to allow the processed material to cool down and harden. A schematic diagram of the mold is reported in FIG. 5. Once the two parts were prepared, 200 mg of acetaminophen were filled into the container cavity. The system was assembled by applying an HPMC aqueous solution onto the contact surface between the elements. The thickness of the capsules shell was 900 microns.

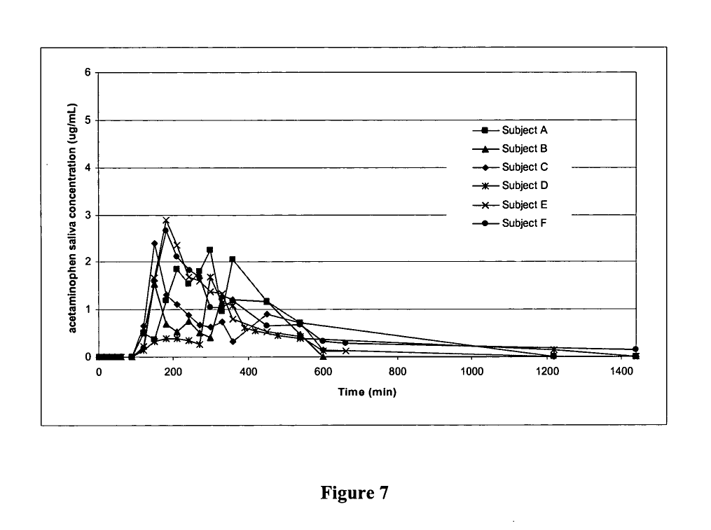

An in vivo study was performed administering one of the above described units to each of six healthy volunteers (age 25-50 years, weight 55-85 Kg). The units were administered to fasted overnight subjects, along with 150 mL water. 3 hours after the administration, a light breakfast was served. 3 mL saliva samples were collected during 24 hours after predetermined time periods. Samples were immediately frozen and stored at −20° C. until analysis. The acetaminophen salivary concentration was determined via HPLC. Drug concentration profiles are reported in FIG. 7.

### EXAMPLE 7

### Composition

| | |
|---|---|
| HPC LF | 90% |
| PEG 1500 | 10% |

The elements of the container were obtained by loading the powder mixture previously prepared in a planetary mixer into an injection molding press (Baby Plast 6/10P, Cronoplast SI). The powder substrate was fed from the hopper into the plasticization unit (165° C.) and then conveyed to the injection unit (160° C.), where it was press-injected through a 2 mm diameter nozzle (160° C.) into the mold cavity within a defined period of time. Before product ejection, the mold was kept closed to allow the processed material to cool down and harden. A schematic diagram of the mold is reported in FIG. 5. Once the two parts were prepared, 200 mg of acetaminophen were filled into the container cavity. The system was assembled and sealed by applying an HPMC aqueous solution onto the contact surface between the elements. The thickness of capsules shell was 1100 microns.

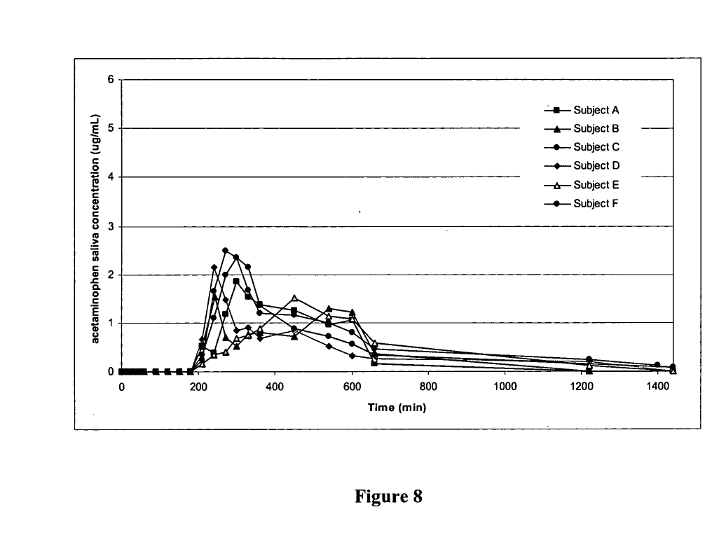

An in vivo study was performed administering one of the above described units to each of six healthy volunteers (age 25-50 years, weight 55-85 Kg). The units were administered to fasted overnight subjects, along with 150 mL water. 3 hours after the administration, a light breakfast was served. 3 mL saliva samples were collected during 24 hours after predetermined time periods. Samples were immediately frozen and stored at −20° C. until analysis. The acetaminophen salivary concentration was determined via HPLC. Drug concentration profiles are reported in FIG. 8.

1. Oral dosage forms for pulsatile release of drugs comprising a container for one or more active ingredients, said container consisting of at least two parts or elements capable of being joined together so as to seal the contents, at least one of said elements being composed of one or more hydrophilic polymers selected from hydroxypropyl methylcellulose (HPMC), hydroxypropylcellulose (HPC), hydroxyethylcellulose (HEC), polyvinylpyrrolidone (PVP), polyvinylalcohol (PVA), polyethylenglycols (PEG), poloxamers (PEO), alginic acid derivatives, and their mixtures, the wall of the elements of the container being of thickness higher than 350 μm.

2. Dosage forms according to claim 1 wherein the container consists of two parts or elements capable of being joined together to delimit an inner closed cavity.

3. Dosage forms according to claim 1 wherein both elements are composed of one or more hydrophilic polymers as defined in claim 1.

4. Dosage forms according to claim 1, wherein the elements are obtained by moulding.

5. Dosage forms according to claim 1, wherein the polymer is selected from hydroxypropyl methylcellulose (HPMC), hydroxypropylcellulose (HPC), polyethylenglycols (PEG), poloxamers (PEO) and their mixtures.

6. Dosage forms according to claim 5 wherein the polymer is hydroxypropylcellulose (HPC).

7. Dosage forms according to claim 1, wherein the wall thickness of the elements of the container is in the 500-1500 μm range.

8. Dosage forms according to claim 1, wherein the elements of the container are joined together by snapping or welding so as to seal the contents.

9. Dosage forms according to claim 1, coated with polymers with pH-dependent solubility, said polymers being soluble only at pH values above 5.

10. A method for manufacturing dosage forms according to claim 1, said method comprising:
shaping the elements of the container by extrusion, wherein said elements are capable of being joined together by snapping or welding so as to seal the contents.

11. The method according to claim 10 wherein said extrusion is accomplished by forcing molten and/or softened masses and/or kneaded products prepared with appropriate binding agents into shaped dies.

12. (canceled)

13. Manufacturing process for dosage forms of claim 1 comprising shaping molten and/or softened masses and/or kneading products prepared with appropriate binding agents by pressure exerted by two optionally heated dies.

14. Dosage forms according to claim 1, wherein said one or more active ingredients are in admixture with pharmaceutically acceptable excipients.

15. Dosage forms according to claim 1, wherein said one or more hydrophilic polymers are are in admixture with pharmaceutically acceptable excipients.

16. Dosage forms according to claim 1, wherein the elements are obtained by injection moulding.

17. A method for manufacturing dosage forms according to claim 1, said method comprising:
layering solutions or dispersions onto inert substrates;
removing solvent from said layered solutions or dispersions to obtain films;
moulding said films to obtain a cavity in which one or more active ingredients are loaded; and
preparing the elements of said container from said molded films.

18. The method of claim 17, wherein said films are obtained by forcing molten and/or softened masses and/or kneaded products prepared with appropriate binding agents through a calender.
